(19) United States
(12) Patent Application Publication
(10) Pub. No.: US 2010/0269932 A1
(43) Pub. Date: Oct. 28, 2010

(54) Resealable Components And Systems

(76) Inventor: Frank M. Richmond, Harvard, IL (US)

Correspondence Address: FITCH EVEN TABIN & FLANNERY, 120 SOUTH LASALLE STREET, SUITE 1600, CHICAGO, IL, 60603-3406, US

(21) Appl. No.: 12/377,290

(22) PCT Filed: Oct. 19, 2006
(86) PCT No.: PCT/US2006/040895

§ 371 Date: Jul. 8, 2010

Related U.S. Application Data
(60) Provisional application No. 60/822,665, filed on Aug. 17, 2006.

(51) Int. Cl. F15D 1/00

(52) U.S. Cl. 137/613

(57) ABSTRACT

The present invention relates to easy to use, cost-effective, resealable components and systems. A disposable device is provided that includes at least one valve that flows in at least one direction, an intermediate structure, and at least one reflux valve. The at least one valve that flows in at least one direction is on a proximal end of the intermediate structure and the at least one reflux valve is on a distal end of the intermediate structure

The present invention relates to easy to use, cost-effective, resealable components and systems. More particularly, a disposable device(s) which includes at least one valve that flows in at least one direction, an intermediate structure, and at least one reflux valve, where the at least one valve that flows in at least one direction is on a proximal end of the intermediate structure and the at least one reflux valve is on a distal end of the intermediate structure.

### BACKGROUND

Patient and health care worker safety are a continuing concern and emphasis in the health care field. Medical preparations or treatments routinely utilize a wide variety of medical components such as for example, intravenous sets, drip chambers, tubings, syringes, pumps, filter, injections sites and various connectors. Any of these medical components may need to be accessed or disconnected multiple times during a medical preparation or treatment regime. Hence, a need exists to insure that these systems can be keep free of contamination over multiples uses.

Health care workers are continually exposed to hazardous materials through needle sticks, transfer of hazardous materials, aerosolization, spills, improper aseptic technique, patient connection (both inadvertent and desired disconnections) and contamination of equipment and work areas. Hence, there is a need to improve the safety of health care workers.

Needle free valve technologies have improved health care worker and patient safety. However, improvement(s) are needed in the area of re-sealable components and systems.

Very recently, various organizations such as NIOSH, ONS, USP 797, and ASHP have recommended going towards closed systems. The configuration of closed systems was not even feasible until development of male reflux valves as described in U.S. Pat. Nos. 6,485,472, 6,206,860, 6,106,502, 6,068,617, 5,848,994, 5,735,826, 5,645,538, 5,476,449, 5,445,630, 5,445,623, 5,405,333, 5,391,150, 5,298,024, 5,022,538, CA 2,171,857 and EP0719158.

### SUMMARY

The present invention is directed to an easy to use, cost-effective, resealable components and systems. The re-sealable system closes upon disconnection, protects against free flow of fluid from the system. Use of the re-sealable system prevents employee contamination, environmental contamination, helps in waste management and control, and prevents contamination of the system.

A disposable device is provided that includes at least one valve that flows in at least one direction, an intermediate structure, and at least one reflux valve. The at least one valve that flows in at least one direction is on a proximal end of the intermediate structure and the at least one reflux valve is on a distal end of the intermediate structure. In this aspect, the valve that flows in one direction may be any type of such valve and may include for example a duck bill valve, umbrella valve, disk valve, spring loaded valve, ball valve, flapper valve and any other means known to control flow in at least one direction. The intermediate structure may include for example containers, fluid lines, tubing, connectors, sets, kits, drip chambers, filters, burette chambers, stopcocks, multiport valves, double ended vials, flow restriction valve, ‘Y’-sites and ‘T’-sites, ambulatory pumps and unions. Further, the reflux valve may be configured as a male reflux valve or female reflux valve with a male or female connection.

Resealable systems and components may be provided in a number of different ways. For example, reflux valves may be placed on various ports of the device or intermediate structure. The reflux valve may be on a proximal end, distal end or both with or without needle or needleless access sites. A syringe, including for example, a pre-filled syringe may be attached through needless access sites including for example “Y” sites, “T” sites and stopcocks. One of ordinary skill in the art would understand that a rubber injection site and needle may be used in place of a needleless access site.

In another aspect, resealable systems may be provided by placing a valve that flows in at least one direction on one or more proximal ports. This valve that flows in at least one direction may be configured to include a male or female shaped connection, with or without an attachment feature.

### BRIEF DESCRIPTION OF THE DRAWINGS

FIG. 1A-B generally describe resealable systems that include combinations of valve(s) that flow in at least one direction (FIG. 1A) with an intermediate structure and reflux valve(s) (FIG. 1B).

FIG. 2 provides some examples of configurations of resealable sets with and without drip chambers.

FIG. 3A-B illustrates configurations of resealable containers.

FIG. 4A-B illustrates configurations of resealable systems that include multiport valves, stopcocks, filters, Y-sites, and T-sites.

FIG. 5 illustrates various configurations of an ambulatory pump.

FIG. 6 shows various configurations of a double-ended vial.

### DETAILED DESCRIPTION

Valves and intermediate structures illustrated in the drawings show various designs. It is understood that any person skilled in the art can choose from a variety of valve(s) that flows in at least one direction, the intermediate structure, and reflux valve(s) in a number of different ways. Various valves and intermediate structures may be attached to each other using methods known in the art such as for example, solvent bonding, RF welding, ultrasonic welding, spin welding, insertion molded, crimping, gluing, soldering, resistance welding, fusion welding, epoxy, and other means known in the art. The valves and intermediate structures may be completely or in part be made out of materials known in the art, plastics, multi-layered plastics, rubber, metal, powdered metal, glass, coated paper, paper materials such as cardboard, foil coated cardboard and laminated cardboard, and any other known materials or any combination of these materials.

Valves that Flow in at Least One Direction

The disposable device includes at least one valve that flows in at least one direction. Examples of valves that flow in at least one direction include duck bills, umbrella, ball valves, spring loaded balls, spring loaded plugs, flapper valves, compression valves, split septum valves, diaphragm and any other valves known in the art which allows flow in at least one direction. Some examples of manufactures of valves that flow in at least one direction include Cardinal, Alaris, B. Braun, Halkey-Roberts, FilterTech, and Bolla.

In another aspect, the valve that flows in at least one direction is activated when an amount of fluid is above the valve in an amount effective for causing the valve to be activated and allow the fluid to flow in at least one direction. This amount of fluid will be equivalent to at least about ⅛ inch (0.3174 cm) of fluid, in another aspect at least about ¼ inch (0.635 cm) of fluid, in another aspect at least about ½ inch (1.27 cm) of fluid, in another aspect at least about ¾ inch (1.905 cm) of fluid, in another aspect at least about 1 inch (2.54 cm) of fluid, in another aspect at least about 1.25 inches (3.175 cm) of fluid, in another aspect at least about 1.5 inches (3.81 cm) of fluid, in another aspect at least about 1.75 inches (4.445 cm) of fluid, in another aspect at least about 2 inches (5.08 cm) of fluid, in another aspect at least about 2.25 inches (5.715 cm) of fluid, in another aspect at least about 2.5 inches (6.35 cm) of fluid, in another aspect at least about 2.75 inches (6.985 cm) of fluid, and in another aspect at last about 3 inches (7.62 cm) of fluid. Containers know in the art will have a length of in excess of 2 inches (5.08 cm) to approximately 15 inches (38.1 cm).

One example of valves that may be utilized includes valves that include a resilient valve member. In this aspect, the resilient valve member defines an outer periphery that is uninterrupted within the periphery. The valve member is deformable to a configuration wherein fluid communication is permitted. In another related aspect, the valve may include a valve member defining an outer periphery that is interrupted at least once within the periphery. The interruption within the periphery allows the fluid to pass directly through the member.

In another aspect, the valve that flows in at least one direction may be activated with a pump such that no amount of fluid above the valve is needed for activation of the valve. Some examples of pumps include those manufactured by Cardinal, Alaris and others known in the art.

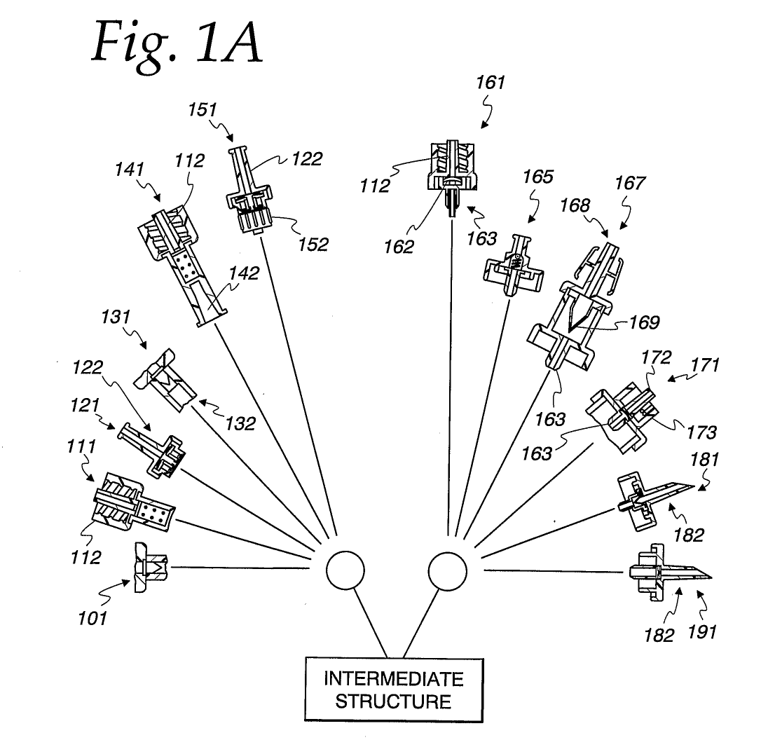

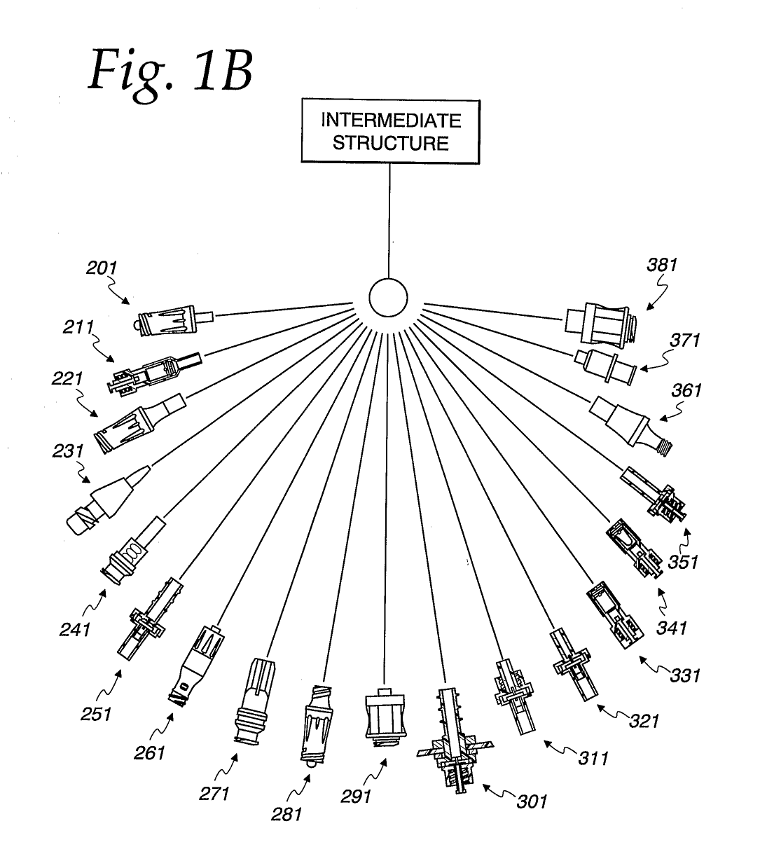

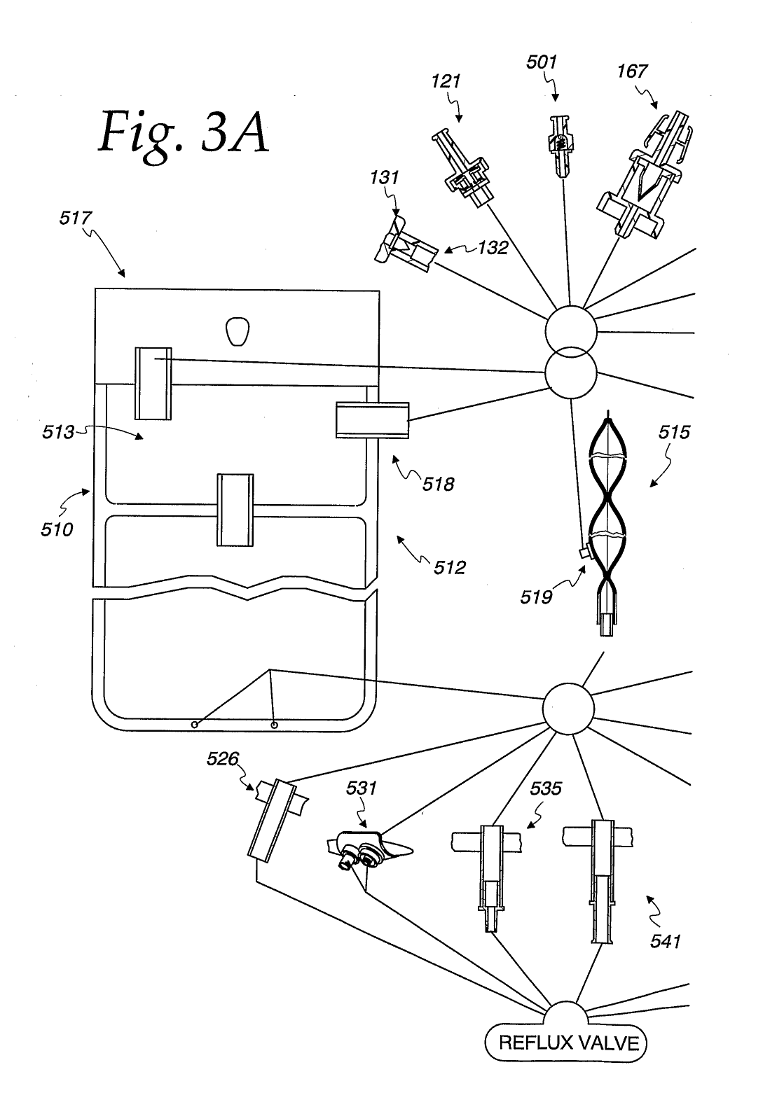

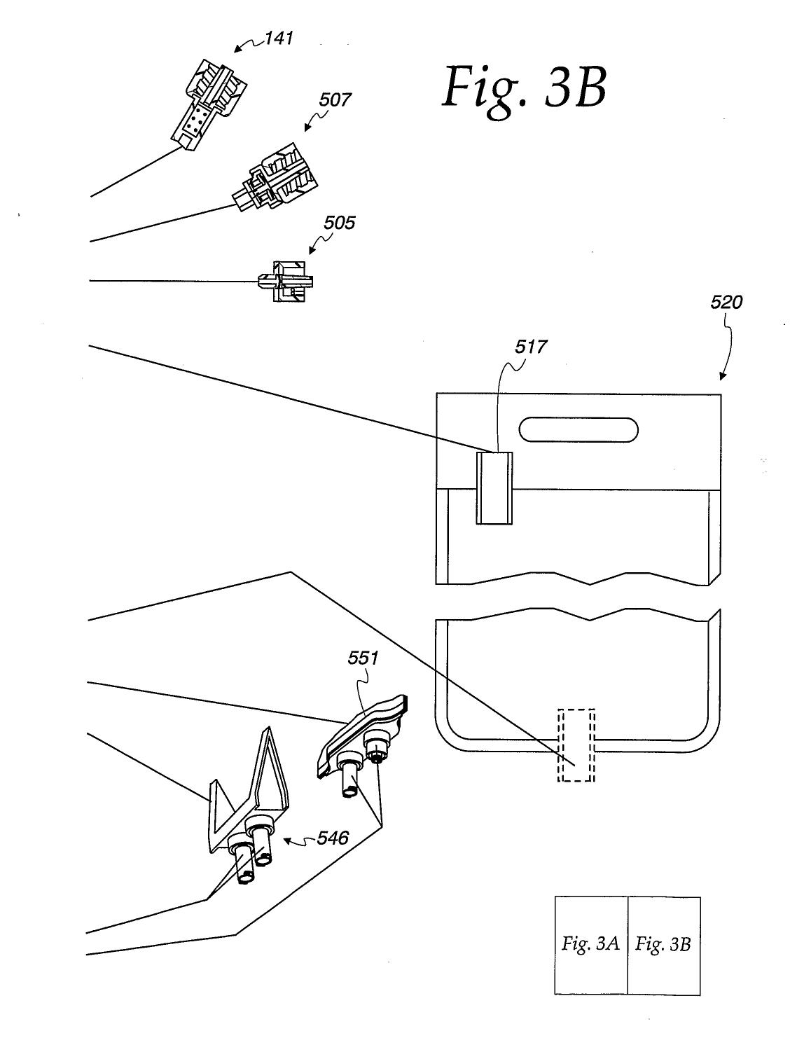

As generally shown in FIG. 1A, the disposable device may include any number of valves that flow in at least one direction on a proximal end(s) of the intermediate structure. Some examples of valves that flow in at least one direction include duck bill valves 101 with male, female or spike connectors where the valve may be solvent bonded to a intermediate structure; duck bill valves 131 with male, female or spike connectors and a tubing pocket 132; Halkey-Roberts style valve 111 with male connector 112 (or any other type of connector not shown); umbrella style valve 121 with a female connector 122 (or any other type of connector not shown); Halkey-Roberts style valve 141 with male connector 112 and female connector 142; umbrella style valve 151 with a female connector 122 and male connector 152; valve 161 with a disc 162, male connector 112, and drip former 163; ball or spring valve 165; valve with hockey post connector 167, cannula 168, duck bill valve 169 and drip former 163; split septum valve 171 with a cannula 172, threaded collar 173 and drip former; duck bill valve 181 with a spike 182; female valve with spring and plug 501 (shown in FIG. 3); male valve with bayonet connections and with a split septum 505 (shown in FIG. 3), a valve with a male connector 507 (shown in FIG. 3) and disk valve 191 with a spike 182. Other valves know in the art may be used.

### Intermediate Structure

The “intermediate structure” referred to in FIG. 1 may include for example, containers, fluid lines, tubing, connectors, sets, kits, drip chambers, drip chambers and sets, filters, burette chambers, stopcocks, multiport valves, double ended vials, flow restriction valve, ‘Y’-sites and ‘T’-sites, sets (for example gravity sets, pump sets, specialty sets and the like), and ambulatory pumps, or valves of any design. Kits may include all of the various components needed for a given procedure. In this aspect, the kit may include at least one and other components known to one of ordinary skill.

### Reflux Valves

As generally shown in FIG. 1B, reflux valves that may be attached to the distal end of an intermediate structure may include any number of configurations. Any male or female reflux valve may be utilized, including for example, reflux valves available from Cardinal, Alaris, B. Braun, Haulkey-Roberts, Filtertec, Bolla, Porex, ICU and other known manufacturers. Some examples of reflux valves include Cardinal female reflux valve 201; Halkey-Roberts male reflux valve 211; swabable female reflux valve 221; B. Braun swabable female reflux valve 231; Porex female reflux valve 241; stepped female reflux valve 251; Alaris swabable remale reflux valve 261; Alaris female reflux valve 271; Alaris male reflux valve 281; Halkey-Roberts swabable female reflux valve 291; a barbed male reflux valve 301; B. Braun style female reflux valve 311; female reflux valve 321; male reflux valve with tamper evident feature 331; Halkey-Roberts male reflux valve 341; disk style male reflux valve 351; ICU swabable reflux valve 361; Halkey-Roberts female reflux valve 371; and Halkey-Roberts swabable reflux valve 381. The female and male reflux valve may include any of those described in U.S. Pat. Nos. 6,485,472, 6,206,860, 6,106,502, 6,068,617, 5,848,994, 5,735,826, 5,645,538, 5,476,449, 5,445,630, 5,445,623, 5,405,333, 5,391,150, 5,298,024, 5,022,538, CA 2,171,857 and EP0719158 which are incorporated herein by reference.

### Disposable Device Configurations

One of ordinary skill will appreciate that valves that flow in at least one direction(s), intermediate devices and reflux valves may be configured in any number of ways. Some examples include but are not limited to combinations of valves with containers, drip chambers, filters, stop cocks, Y-sites, T-sites, and any disposable or medical system that has at least one proximal and at least one distal connection.

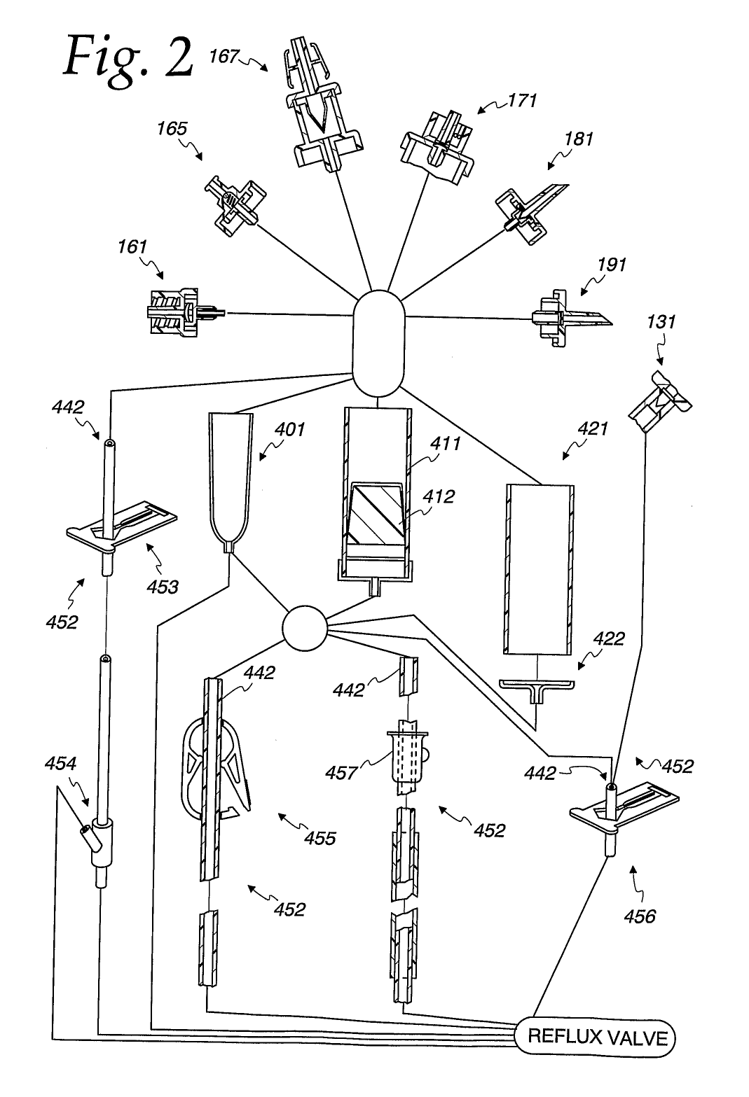

FIG. 2 provides some examples of configurations of sets and drip chambers. As illustrated, any one of the at least one way valves 161, 165, 167, 171, 181, 191 or any other type of at least one way valve may be configured on the proximal end of a drip chamber. The drip chamber can be for example a one piece drip chamber body 401, a drip chamber 411 with a filter 412, or a drip chamber 421 with a detachable end cap 422, and other designs of drip chambers known in the trade. Drip chamber may include either a vent, filter or combination of any of the above. The drip chamber may be connected further to a tubing and/or other components which forms a set 452. The set 452 may include a clamp such as 455 or 456 or any other style of clamp/flow restrictor know in the art, and may further include a flow control valve 457. The sets 452 are then configured to include any reflux valve, such as for example as illustrated in FIG. 1B.

In another aspect as shown in FIG. 2, anyone of the at least one way valves 161, 165, 167, 171, 181, 191 or any other type of at least one way valve may be configured through a tubing connector to a tube 442 which is part of a set 452. The set 452 may include for example a clamp 453 and Y-site 454. The set 452 may then be configured to include any reflux valve, such as for example as illustrated in FIG. 1B.

As further shown in FIG. 2, a duck bill valve may be connected directly to a set 452. The duck bill valve may be configured with a female of male connector or may be configured with a spike.

FIGS. 3A-B shows some configurations of containers, such as for example a medicament bag. As illustrated, any one of the at least one way valves 121, 131, 141, 167, 401, 505, 507 or any other type of at least one way valve may be configured on the proximal end or any other location of a container, such as for example top seam(s), side seam(s), and belly button port. Any of the at least one way valves may be connected directly to the container by known methods or be connected to the container by a tubing connection.

The container may be a medicament container 520, a multi-material medicament container 515 or a medicament container 510 with a tear seal 512 or a medicament container with a frangible 513. Any of the at least one valve that flow in at least one direction may be connected to the container or bag in a number of locations. For example, a valve that flows in at least one direction may be at a proximal end 517, at a side proximal end 518, or at a side 519 of the container.

All of the containers may include any number of at least one or more different ports at their distal ends. For example, the container may include a straight through port 526, a saddle port 531, a straight through male port 535, a straight through female port 541, a wedge port 546, or a boat port 551. The ports at the distal ends of the container(s) may be connected to any known configuration of reflux valve, including for example, those shown in FIG. 1B. Alternatively, a reflux valve may be connected directly to a distal end of the container.

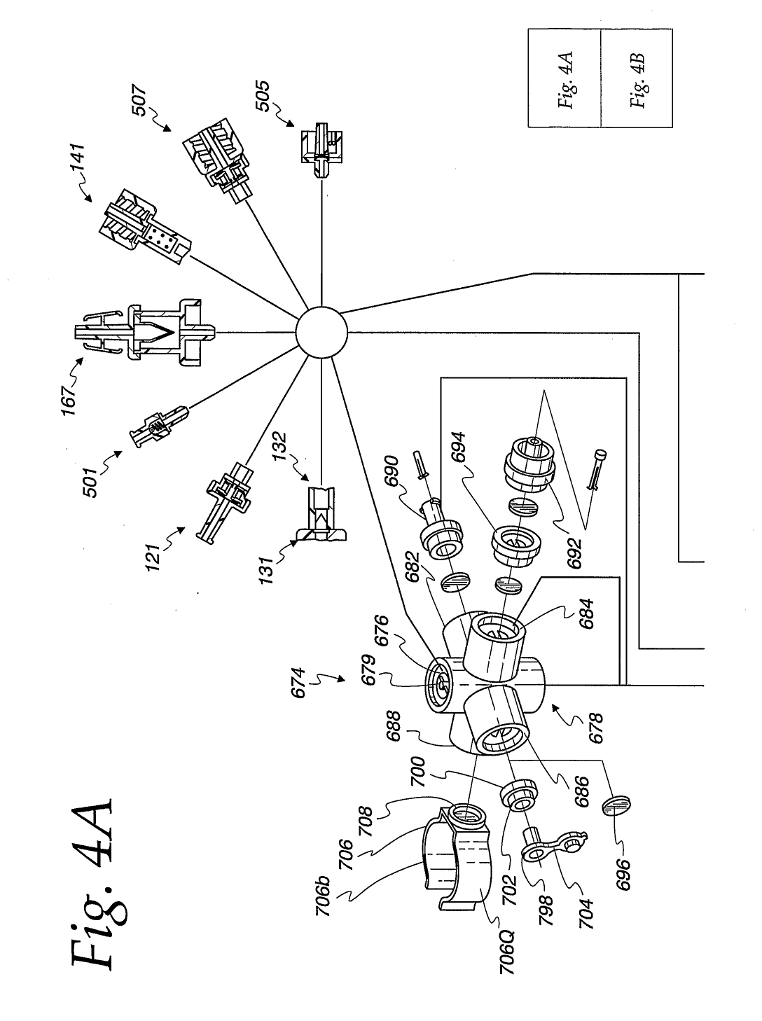

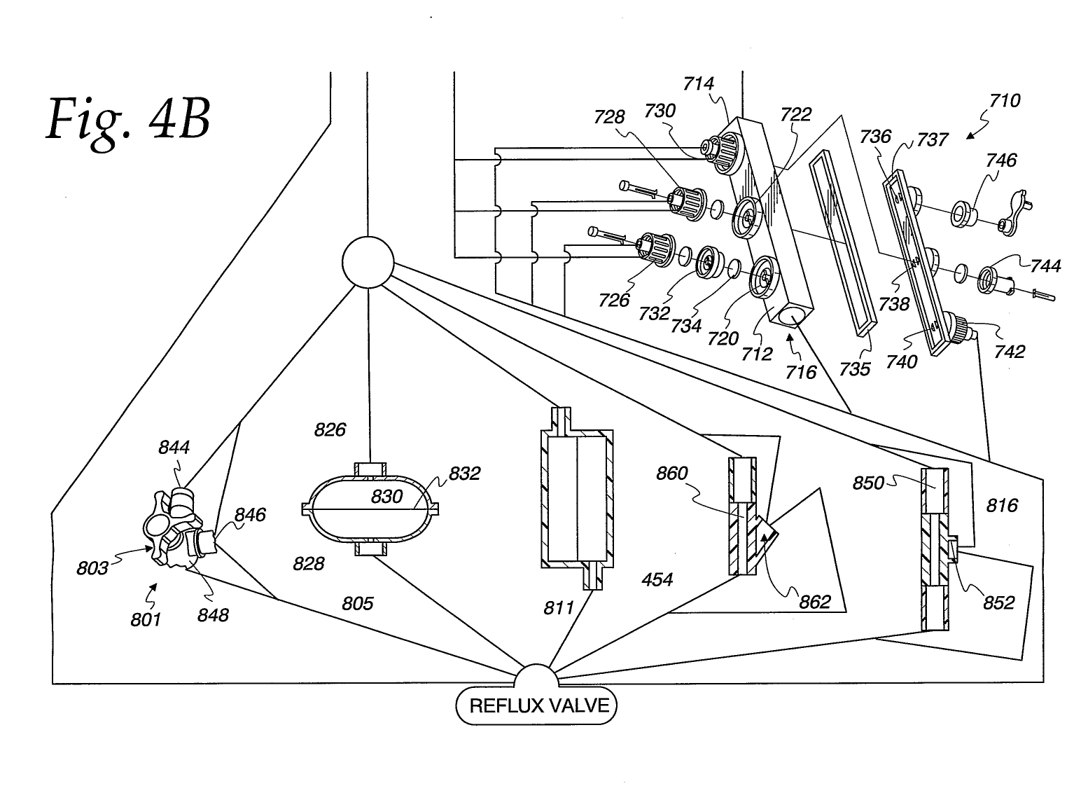

Now referring to FIGS. 4A-B, configurations including multiport valves, stopcocks, filters. Y-sites and T sites can be seen. As illustrated in FIG. 4, any one of the valves that flows in at least one direction 121, 131, 141, 167, 401, 505, 507 or any other type of valve that flows in at least one direction may be configured to multiport valve(s) 674 and 710.

As shown, the multiport valve 674 includes a first port 676 which is generally cylindrically shaped, and a second port 678 which is also generally cylindrically shaped and which may be configured as a female luer fitting or any other type of connection. As can be appreciated in reference to FIG. 4, the first and second ports 676, 678 are coaxial and establish a main fluid passageway therebetween. As shown, a valve that flows in at least one direction may be connected to the multiport valve 674 at the first port 676. Alternatively, the valve that flows in at least one direction may be at any one or more of the third 682, fourth 684, fifth 686 and sixth 688 ports described below.

FIGS. 4A-B additionally shows that the multiport valve 674 includes third, fourth, fifth and sixth ports 682, 684, 686, 688, all of which are generally cylindrically shaped. As shown, the third and fifth ports 682, 686 are coaxial with each other. Likewise, the fourth and sixth ports 684, 688 are coaxial with each other. Each of the third through sixth ports 682, 684, 686, 688 defines a respective fluid pathway, and fluid communication through the fluid pathway can be selectively established or otherwise effected as valve member disclosed below.

For example, a female reflux valve 690 can be disposed in the third fluid port 682 for selectively establishing fluid communication through the port 682 and into the main fluid passageway 679 in accordance with principles valve member disclosed previously. Moreover, a combination male reflux valve-valve that flows in at least one direction 692, 694, can be disposed in the fourth fluid inlet port 684 of the multiport valve 674. If desired, the male reflux valve 692 can be replaced with a female reflux valve (not shown).

Additionally, fluid communication through the fifth inlet port 686 can be permanently blocked if desired by bonding a plug 696 within the port 686 by means well known in the art. Alternatively, the fifth fluid inlet port 686 can hold a fluid filter, e.g. a filter 698. As shown, the filter 698 includes a filter element 700 having a membrane 702 through which air can pass. The filter 698 also includes a plug element 704 which engages filter element 700 and which holds the filter element 700 within the sixth fluid port 686. It is understood that anyone skilled in the art could use any combination of the above.

An inline multiport valve 710 is also shown in FIGS. 4A-B. As shown, the valve 710 includes an elongated, generally parallel-piped-shaped valve body 712 that is formed with a fluid inlet port 714 and a fluid outlet port 716. The fluid inlet port 714 may be connected with any valve that flows in at least one direction. Alternatively, the inlet port 714 may instead be any one of the other inlet ports on the multiport valve 710. A main fluid passageway is established through the valve body 712 between the ports 714, 716.

Additionally, the valve body 712 is formed with two to six fluid inlet ports. In this aspect shown in FIG. 4, the valve body 712 includes first through third fluid inlet ports 720, 722, 724. Further, one or more reflux valves may be positioned in the ports 720, 722, 724 to selectively establish fluid communication therethrough. More particularly, a first male reflux valve 726 is positioned in the first port 724, a second male reflux valve 728 is positioned in the second port 726, and a third male reflux valve 730 is positioned in the third port 724. The first male reflux valve 726 can include a valve that flows in at least one direction 732 having a deformable valve member 734.

When the inline multiport valve 710 includes only three ports, a solid, continuous parallel-piped-shaped bottom plate 735 is rf sealed or ultrasonically welded to the valve body 712 opposite the first through third ports 720, 722, 724. On the other hand, the inline multiport valve 710 may include additional ports, in which case the bottom plate 735 is replaced with a port plate 737 which is formed with fourth through sixth ports 736, 738, 740. Although it is understood by a person skilled in the art that there could be any number of ports. A male reflux valve 742 can be positioned in the sixth port 740, a female reflux valve 744 can be positioned in the fifth port 738, and a filter 746 which is similar to the filter 698 can be positioned in the fourth port 736. The fourth port 736 can include the mounting bracket, reflux valves, filter, plug, and check valve options. Either multipart valve 674 and 710 may then be connected to a reflux valve shown in FIG. 1B through any remaining port.

As further illustrated in FIGS. 4A-B, a valve that flows in at least one direction may also be configured to a stopcock 801, filter 805, filter 811, Y-site 454 and T-site 816. Any one of these components may be connected to a reflux valve as shown in FIG. 1B.

In accordance with principles well known in the art, the stopcock 801 includes a central fluid passageway that is covered by a cover plate (not shown), and a means for directing flow 803. Additionally, the stopcock 801 can include at least two ports, and may include a third and/or fourth port. The stopcock 801 as shown in FIG. 4 includes first, second and third ports 844, 846, and 848. A valve that flows in at least one direction is disposed in a proximal port and may be in one or two other ports other than a distal port. A reflux valve is disposed in a distal end and may be in one or two of the remaining ports. The stopcock, Y-site and T-site could include at least one proximal end with a solvent bond attachment.

Further referring to FIGS. 4A-B, a filter chamber 805 includes a first port 626, a second port 828, and a chamber 630 formed therebetween. As shown, the chamber 830 is catenary-shaped in cross section. Also, a disk-shaped filter 832 is disposed in the catenary-shaped chamber 830. A valve that flows in at least one direction may be disposed in either the first port 826 or second port 828. The opposite (at least one distal) port is configured with a reflux valve(s). As will be appreciated by one of ordinary skill, the filter may be a filter 805, a filter 811, or any other type of known filter.

FIGS. 4A-B also shows a Y-site connector 454 configured with a valve that flows in at least one direction and a reflux valve. As shown, the Y-site connector 454 defines a main cylindrical fluid passageway 860 and a secondary fluid passageway 862. A valve that flows in at least one direction may be disposed in any one of the proximal ports on the Y-site connector. At least one of the remaining ports (distal) will then be configured with a reflux valve.

FIGS. 4A-B further shows a T-site connector 816 configured with a valve that flows in at least one direction and a reflux valve. The T-site connector 816 defines a main fluid passageway 850 and a secondary fluid passageway 852. A valve that flows in at least one direction may be disposed in any one of the proximal ports on the T-site connector. At least one of the remaining port (distal) will then be configured with a reflux valve.

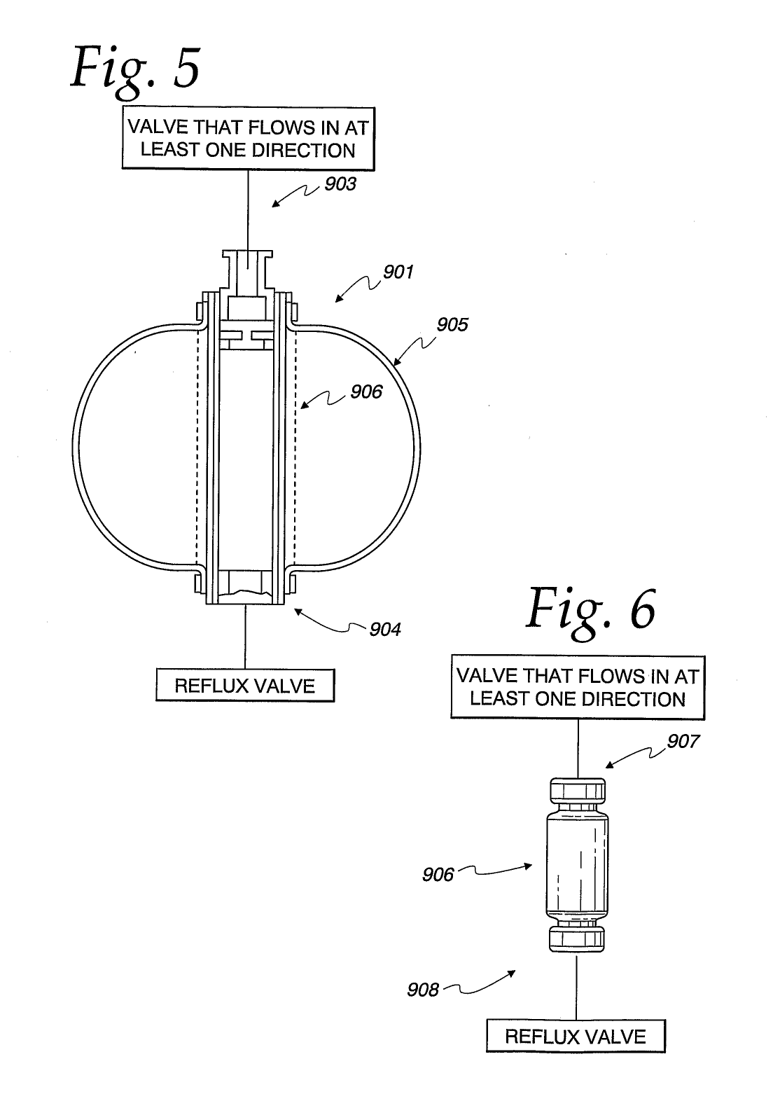

FIG. 5 illustrates various configurations of an ambulatory pump. The ambulatory pump 901 includes a valve that flows in at least one direction at its proximal end 903 and a reflux valve at it distal end 904. Some examples of valves that flow in at least one direction are shown in FIG. 1A and some examples of reflux valves are shown in FIG. 1B. The ambulatory pump may include a membrane which expands when filled (as shown by 905) and contracts when empty (as shown by 906).

FIG. 6 shows various configurations of a double ended vial. The double ended vial 906 includes a valve that flows in at least one direction at its proximal end 907 and a reflux valve at it distal end 908. Some examples of valves that flow in at least one direction are shown in FIG. 1A and some examples of reflux valves are shown in FIG. 1B.

Numerous modifications and variations in practice of the invention are expected to occur to those skilled in the art upon consideration of the foregoing detailed description of the invention. Consequently, such modifications and variations are intended to be included within the scope of the following claims.

What is claimed is:

1. A disposable device comprising at least one valve that flows in at least one direction, an intermediate structure, and at least one reflux valve, wherein the at least one valve that flows in at least one direction is on a proximal end of the intermediate structure and the at least one reflux valve is on a distal end of the intermediate structure.

2. The disposable device of claim 1 wherein the reflux valve is a male reflux valve.

3. The disposable device of claim 2 where the reflux valve is a male luer reflux valve.

4. The disposable device of claim 1 wherein the reflux valve is a female reflux valve.

5. The disposable device of claim 4 where the reflux valve is a female luer reflux valve.

6. The disposable device of claim 1 wherein the intermediate structure is selected from the group consisting of containers, fluid lines, tubing, connectors, sets, kits, drip chambers, drip chambers and sets, filters, burette chambers, stopcocks, multiport valves, double ended vials, flow restriction valve, ‘Y’-sites and ‘T’-sites, ambulatory pumps and unions.

7. The disposable device of claim 1 wherein the valve that flows in at least one direction is selected from the group consisting of duck bills, umbrella, ball valves, spring loaded balls, spring loaded plugs, flapper valves, compression valves, split septum valves, and diaphragm valves.

8. The disposable device of claim 1 wherein the valve that flows in at least one direction includes a male connector, female connector, or a spike.

9. A disposable device comprising at least one valve that flows in at least one direction on a proximal end of drip chamber and at least one reflux valve on a distal end of a drip chamber.

10. The disposable device of claim 9 wherein the valve that flow in at least one direction is selected from the group consisting of duck bills, umbrella, ball valves, spring loaded balls, spring loaded plugs, flapper valves, compression valves, split septum valves, and diaphragm valves.

11. The disposable device of claim 9 wherein the reflux valve is a male reflux valve.

12. The disposable device of claim 11 where the reflux valve is a male luer reflux valve.

13. The disposable device of claim 9 wherein the reflux valve is a female reflux valve.

14. The disposable device of claim 13 where the reflux valve is a female luer reflux valve.

15. The disposable device of claim 9 wherein the valve that flows in at least one direction includes a male connector, female connector, or a spike.

16. A method for providing a resealable disposable device comprising:
providing an intermediate structure with a valve that flows in at least one direction at a proximal end of the intermediate structure; and
providing the intermediate structure with at least one reflux valve at a distal end of the intermediate structure.

17. The method of claim 16 wherein the reflux valve is a male reflux valve.

18. The method of claim 17 where the reflux valve is a male luer reflux valve.

19. The method of claim 16 wherein the reflux valve is a female reflux valve.

20. The method of claim 19 wherein the reflux valve is a female luer reflux valve.

21. The method of claim 16 wherein the intermediate structure is selected from the group consisting of containers, fluid lines, tubing, connectors, sets, kits, drip chambers, drip chambers and sets, filters, burette chambers, stopcocks, multiport valves, double ended vials, flow restriction valve, ‘Y’-sites and ‘T’-sites, ambulatory pumps and unions.

22. The method of claim 16 wherein the valve that flows in at least one direction is selected from the group consisting of duck bills, umbrella, ball valves, spring loaded balls, spring loaded plugs, flapper valves, compression valves, split septum valves, and diaphragm valves.

23. The method of claim 16 wherein the valve that flows in at least one direction includes a male connector, female connector, or a spike.
